(19) United States
(12) Patent Application Publication
(10) Pub. No.: US 2023/0233732 A1
(43) Pub. Date: Jul. 27, 2023

(54) STERILIZATION AIR CONDITIONER

(71) Applicants: Quanling WANG, Qinhuangdao (CN); Miaohong WANG, Qinhuangdao (CN)

(72) Inventors: Quanling WANG, Qinhuangdao (CN); Miaohong WANG, Qinhuangdao (CN)

(21) Appl. No.: 18/190,792

(22) Filed: Mar. 27, 2023

Related U.S. Application Data
(63) Continuation of application No. PCT/CN2020/124601, filed on Oct. 29, 2020.

(30) Foreign Application Priority Data
Sep. 27, 2020 (CN) 202011033170.1

(51) Int. Cl. A61L 9/20; A61L 9/22; A61L 9/14; F24F 3/16

(52) U.S. Cl. CPC A61L 9/20; A61L 9/22; A61L 9/145; F24F 3/16; A61L 2209/16; A61L 2209/134; A61L 2209/132

(57) ABSTRACT

A sterilization air conditioner is provided in this disclosure, which includes an indoor unit and a sterilization device. The sterilization device is arranged on the indoor unit, and the sterilization device is configured for sterilizing the air entering the indoor unit and a body of the indoor unit by releasing chemical or physical media. In view of a structure of the air conditioner, a front end of a filter screen of the air conditioner is equipped with a disinfectant dripping, drenching, sprinkling, spraying or atomizing or a plasma or ultraviolet or electrostatic negative oxygen ion sterilization device, and disinfectant or plasma or ultraviolet or negative oxygen ion is sprayed to the air conditioner for sterilization. The disclosure has advantages of simplicity, convenience and low investment, and is more suitable for sterilization of household split air conditioners and central air-conditioning fan-coil air conditioners.

### CROSS-REFERENCE TO RELATED APPLICATIONS

This application is a continuation of International Patent Application No. PCT/CN2020/124601 with a filing date of Oct. 29, 2020, designating the United States, now pending, and further claims priority to Chinese Patent Application No. 202011033170.1 with a filing date of Sep. 27, 2020. The content of the aforementioned applications, including any intervening amendments thereto, are incorporated herein by reference.

### TECHNICAL FIELD

The disclosure relates to the technical field of air conditioners, in particular to a sterilization air conditioner.

### BACKGROUND ART

Air conditioners in China have entered hundreds of millions of families, bringing us warmth and coolness; but at the same time, the air conditioners have also caused pollution to us. According to surveys of air conditioning hygiene by China's Center for Disease Control and Prevention, only 6.12% of air conditioners are qualified at present, and almost half of remaining air conditioners are seriously polluting ones and almost the other half of them are heavily polluting ones.

According to operation principle of refrigeration of the air conditioners, dew is condensed at a surface air cooler of an indoor unit for a long time, which results in low temperature and humidity inside the surface air cooler of the indoor unit and a cabinet, which is very suitable for growth and reproduction of bacteria and viruses, especially for survival and reproduction of Corona Virus Disease 2019 virus, which can be breathed into lungs of persons and also can be blown to any corner of and any object in a house through an air-conditioning ventilation system. In addition, a primary filter is provided in front of the surface air cooler, air enters the surface air cooler through a filter screen, and dust in the air is gathered on the filter screen after long-term operation, and thus germs are more likely to survive and reproduce in the filter screen. Therefore, people who often stay in air-conditioned rooms often get sick. Harms may be caused to human functions such as a nervous system, a respiratory system and an immune system for people who stay in the air-conditioned rooms for a long time. Therefore, it is not an exaggeration that the air conditioners are gradually called a chronic killer at home and abroad.

Although an ultraviolet lamp tube can serve to sterilize, only parts that can be directly irradiated by ultraviolet rays can be sterilized and parts that can't be irradiated can't be sterilized because the ultraviolet rays can't diffract. Heating the surface air cooler to 56° C. for sterilization is only aimed at the surface air cooler itself, especially the air conditioner has an air inlet and an air outlet, which cannot be sealed. Therefore, not every part of the indoor unit of the air conditioner, especially the filter screen of the filter cannot be heated to a temperature of 56° C., and it is impossible to keep a time space for sterilization at 56° C. for 30 minutes. In addition, the temperature of 56° C. is only intended to kill a few bacteria and viruses, so above sterilization at 56° C. can't guarantee complete inactivation, which can't be called sterilization.

The sterilization refers to a measure that uses strong physical and chemical factors to make all microorganisms inside and outside any object lose their growth and reproduction ability forever. Commonly used sterilization methods include chemical reagent sterilization, ray sterilization, dry heat sterilization, moist heat sterilization and filtration sterilization. Thoroughness of sterilization is restricted by sterilization duration and strength of sterilization agent. Resistance of microorganisms to sterilant depends on original population density, or resistance of strains or given to the strains by an environment. Effect of the sterilization refers to physical or chemical methods to kill all microorganisms, including pathogenic and non-pathogenic microorganisms and spores, so that they can reach a sterility assurance level. After the sterilization, an uncontaminated item is called a sterile item. After the sterilization, an uncontaminated area is called a sterile area. The sterilization refers to killing or eliminating all microorganisms in a propagation medium, including pathogenic microorganisms and non-pathogenic microorganisms, and also including bacterial spores and fungal spores. Sterilization is a process to make products free of survived microorganisms, a goal of which is to kill or remove vegetative cells of all microorganisms and spores (or conidia) thereof in materials, so as to achieve a sterile process.

Dry heat sterilization. Burning sterilization or hot air sterilization in oven is called the dry heat sterilization.

Metal instruments or washed glassware can be completely sterilized (including bacterial spores) after being placed in an electric oven at 150 to 170° C. for 1 to 2 hours. Burning is the most thorough dry heat sterilization method, with its application scope being limited to sterilization of inoculating loops and needles or burning of materials with pathogenic bacteria and animal carcasses. This method is not suitable for sterilization for the air-conditioning system because of its high temperature.

Moist heat sterilization. Sterilization is made with boiling water, vapor or vapor pressurizing. Pasteurization is the moist heat sterilization, in which there are two low temperature holding method: (1) treatment at 61.7 to 62.8° C. for 30 minutes. (2) A modern high temperature short time method or treatment at 71.6° C. or slightly higher temperature for 15 minutes. Among above methods, the vapor pressurizing sterilization presents optimum effect, which can be made by vapor at a normal pressure or in a high-pressure vapor pot (generally at 1 kg/cm²), with a vapor temperature up to 121° C., which can kill all heat-resistant spores in 30 minutes. However, some substances that are easily destroyed by high pressure, such as some sugars or organic nitrogen compounds, and sterilization is optimally made at a pressure of 0.6 kg/cm² at 110° C. for 15 to 30 minutes; at 135 to 145° C. for 3 to 5 hours; at 160° C. to 170° C. for 2 to 4 hours; 180 to 200° C. for 0.5 to 1 hour; and at above 200° C. for more than 0.5 hours. This method is not suitable for sterilization for the air-conditioning system because of its high temperature and the boiling water and the vapor.

Chemical reagent sterilization. Most chemical reagents have antibacterial effect at a low concentration and sterilization effect at a high concentration. 5% of carbolic acid, 70% of ethanol and ethylene glycol are commonly used. Chemical sterilants must be volatile in order to remove residual drugs on sterilized materials. Commonly used reagents for chemical sterilization include surface disinfectants, antimetabolites (sulfonamides, etc.), antibiotics and biopharmaceuticals. The antibiotics are a kind of secondary metabolites or artificial derivatives synthesized during life activities of microorganisms or other organisms, and they can inhibit life activities of or infect other organisms (including pathogens, viruses, cancer cells, etc.) at a very low concentrations, so that they can be used as excellent chemotherapeutic agents. The chemical reagent sterilization is more suitable for sterilization for the air-conditioning system because of its low temperature.

Bacteria blocking by filter. The filter screen is configured to block the bacteria, in which the bacteria cannot be inactivated. The bacteria is blocked on the filter screen for a long time, can't be inactivated and multiply on the filter screen, which is also very dangerous and harmful to people in the air-conditioned rooms. A virus has a smallest particle diameter, and common viruses have a diameter of 0.01 to 0.02 with a largest pox virus in animals having a diameter of 0.17 to 0.26 some large one having a diameter of 0.3 to 0.45 u m, and a foot and mouth disease virus having a diameter of 0.01 u m, which is the smallest virion in the world at present. For common influenza viruses, an influenza A HIN1 virus is 0.09 u m in diameter, an Ebola virus is 0.08 μm in diameter, a Sars virus is 0.06 to 0.22 u m in diameter and a Corona Virus Disease 2019 virus is 0.06 to 0.14 u m in diameter. Diameters of most bacteria range from 0.5 to 5 u m, with smallest bacteria of 0.2 μm in diameter. To sum up, a HEPA high-efficiency filter can only block bacteria of more than or equal to 0.3 u m instantly, and can't block bacteria of less than or equal to 0.3 u m, let alone viruses of 0.01 to 0.3 μm. To sum up, the HEPA high-efficiency filter can only be used to block the bacteria, but not viruses, which cannot be called sterilization.

### SUMMARY

A first object of the present disclosure is to provide a sterilization air conditioner, which can solve a problem of inactivating viruses and bacterial spores.

A sterilization air conditioner is provided in this disclosure, which includes an air conditioning system terminal and a sterilization device.

The sterilization device is arranged on the air conditioning system terminal, and is configured for sterilizing air entering the air conditioning system terminal and the air conditioning system terminal.

Preferably, the air conditioning system terminal includes an indoor unit or a fan coil.

The indoor unit includes a split-type wall-mounted air conditioning unit, a cabinet and a ceiling-concealed indoor unit.

The fan coil includes a surface mounted fan coil and a concealed fan coil.

Preferably, the sterilization device includes a physical sterilization device and/or a chemical sterilization device.

The chemical sterilization device is configured to sterilize by releasing disinfectant by a dripping, drenching, sprinkling, spraying or atomizing device.

The physical sterilization device is configured to sterilize by releasing plasma or ultraviolet rays or negative oxygen ions.

Preferably, the disinfectant includes hydrogen peroxide or hydrogen peroxide silver ion or chlorine dioxide or formaldehyde or glutaraldehyde or ethylene oxide or peracetic acid or chlorine or hypochlorous acid or copper sulfate or ethanol or isopropanol or n-propanol disinfectant.

Preferably, the sterilization device includes a disinfectant storage tank, disinfectant and a disinfectant ejection port arrangement.

The disinfectant is stored in the disinfectant storage tank, the disinfectant ejection port arrangement (10) is communicated with the disinfectant, the disinfectant ejection port arrangement is arranged inside or outside the air inlet of the air conditioning system terminal, and the disinfectant ejection port arrangement includes at least one ejection port.

The sterilization device is arranged outside the air conditioning system terminal or inside the air conditioning system terminal.

Preferably, the sterilization device includes at least one group of ultrasonic disinfectant atomizing devices.

Each of at least one group of ultrasonic disinfectant atomizing devices includes a disinfectant storage tank, disinfectant, an ultrasonic generator and an ultrasonic electric control device.

The ultrasonic generator is immersed within the disinfectant, the ultrasonic disinfectant atomizing device is communicated with the disinfectant ejection port arrangement, the ultrasonic electric control device is connected with the ultrasonic generator, and the disinfectant ejection port arrangement is arranged inside or outside the air inlet of the air conditioning system terminal.

The sterilization device is arranged outside the air conditioning system terminal or inside the air conditioning system terminal.

Preferably, the sterilization device includes at least one group of ultrasonic disinfectant atomizing devices.

Each of the at least one group of ultrasonic disinfectant atomizing devices includes a storage tank, disinfectant, an ultrasonic generator, an ultrasonic electric control device and a disinfectant atomizing spray pipe.

The ultrasonic generator is arranged outside the disinfectant and the ultrasonic generator is communicated with the disinfectant. The disinfectant atomizing spray pipe is communicated with the disinfectant ejection port arrangement, the ultrasonic electric control device is connected with the ultrasonic generator, and the disinfectant ejection port arrangement is arranged inside or outside the air inlet of the air conditioning system terminal.

The sterilization device is arranged outside the air conditioning system terminal or inside the air conditioning system terminal.

Preferably, the sterilization device includes a disinfectant storage tank, disinfectant, a disinfectant pump and at least one nozzle.

The disinfectant pump is communicated with the disinfectant, the nozzle is connected with the disinfectant pump, and the nozzle is arranged inside or outside an air inlet of an indoor cabinet of the air conditioner at the air conditioning system terminal.

The sterilization device is arranged outside the air conditioning system terminal or inside the air conditioning system terminal.

Preferably, the sterilization device includes an ultrasonic disinfectant atomizing device, a disinfectant atomizing and blasting pipe and at least one disinfectant atomizing and jetting port.

The disinfectant atomizing and blasting pipe is communicated with the disinfectant atomizing spray pipe, the disinfectant atomizing and jetting port is connected with the disinfectant atomizing and blasting pipe, and the disinfectant atomizing and jetting port and the disinfectant atomizing and blasting pipe are arranged inside or outside the air inlet of the indoor cabinet of the air conditioner at the air conditioning system terminal.

The sterilization device is arranged outside the air conditioning system terminal or inside the air conditioning system terminal.

Preferably, the sterilization air conditioner includes a sterilization device, an air inlet of the air conditioning system terminal and an inlet-air filter screen.

The sterilization device includes a storage tank, disinfectant, at least one ultrasonic disinfectant atomizing device, an ultrasonic generator, an ultrasonic electric control device, a disinfectant atomizing spray pipe and at least one disinfectant atomizing and jetting port.

The ultrasonic generator is communicated with the disinfectant, the ultrasonic generator is connected with the ultrasonic electric control device, and the disinfectant atomizing spray pipe is communicated with the disinfectant atomizing and jetting port.

The ultrasonic disinfectant atomizing device is at least arranged on one inlet-air filter screen.

The sterilization device is arranged outside the air conditioning system terminal or inside the air conditioning system terminal.

Preferably, the sterilization device includes at least one dripping device, and the dripping device includes a disinfectant storage tank, a disinfectant and a dripping device.

The dripping device is communicated with the disinfectant, and the dripping device can be a valve or a medical drip control valve and is configured for dripping disinfectant liquid to at least one inlet-air filter screen.

The sterilization device is arranged outside the air conditioning system terminal or inside the air conditioning system terminal.

Preferably, the sterilization device comprises an ultrasonic disinfectant atomizing device.

The ultrasonic disinfectant atomizing device includes a storage tank, disinfectant, an ultrasonic generator, an ultrasonic electric control device, a disinfectant atomizing spray pipe, at least one disinfectant atomizing and blasting pipe and at least one disinfectant atomizing and jetting port.

The disinfectant atomizing and blasting pipe is communicated with the disinfectant atomizing spray pipe, the disinfectant atomizing and jetting port is connected with the disinfectant atomizing and blasting pipe, the disinfectant atomizing and jetting port and the disinfectant atomizing and blasting pipe are arranged inside or outside the air inlet of the air conditioning system terminal, the ultrasonic generator is arranged outside the disinfectant, the ultrasonic generator is communicated with the disinfectant, and the ultrasonic electric control device is connected with the ultrasonic generator.

The sterilization device is arranged outside the air conditioning system terminal or inside the air conditioning system terminal.

Preferably, a shell of the indoor unit and/or a panel of an inlet-air filter screen are of a structure with a cavity, and the shell with the cavity constitutes the storage tank and stores the disinfectant.

Preferably, the sterilization air conditioner includes a fan-coil air conditioner, a fan-coil air inlet and a return air inlet of the fan-coil air conditioner.

The sterilization device includes a disinfectant storage tank, disinfectant, a disinfectant pump and at least one nozzle.

The fan-coil air conditioner is arranged in a ceiling of an air-conditioned room, the disinfectant storage tank is arranged in or outside the ceiling of the air-conditioned room, and the return air inlet of the fan-coil air conditioner is arranged on the ceiling of the air-conditioned room.

The disinfectant pump is connected with the disinfectant storage tank, the disinfectant pump is communicated with the disinfectant, and the nozzle is connected with the disinfectant pump. The nozzle is arranged at an air inlet of the fan-coil air conditioner or at the return air inlet of the fan-coil air conditioner, or inside or outside the air return outlet of the fan-coil air conditioner.

Preferably, the sterilization air conditioner includes a fan-coil air conditioner, a fan-coil air inlet and a return air inlet of the fan-coil air conditioner.

The sterilization device includes an ultrasonic disinfectant atomizing device, a disinfectant atomizing and blasting pipe and at least one disinfectant atomizing and jetting port.

The ultrasonic disinfectant atomizing device includes a disinfectant storage tank, disinfectant, an ultrasonic generator, an ultrasonic electric control device and a disinfectant atomizing spray pipe.

The ultrasonic generator is arranged outside the disinfectant and is communicated with the disinfectant, the ultrasonic electric control device is connected with the ultrasonic generator, the disinfectant atomizing and blasting pipe is communicated with the disinfectant atomizing and jetting port, and the disinfectant atomizing and blasting pipe is connected with the disinfectant atomizing spray pipe.

The fan-coil air conditioner is arranged in a ceiling of an air-conditioned room, and the return air inlet of the fan-coil air conditioner is arranged on the ceiling of the air-conditioned room.

The disinfectant atomizing and blasting pipe is arranged inside or outside the air inlet of the fan-coil air conditioner, or inside or outside the return air inlet of the fan-coil air conditioner, the disinfectant atomizing and blasting pipe is connected with the disinfectant atomizing spray pipe, the disinfectant atomizing and jetting port is communicated with the disinfectant atomizing and blasting pipe, and the ultrasonic disinfectant atomizing device is arranged inside or outside the ceiling of the air-conditioned room.

Preferably, the air conditioning system terminal includes a sterilization device and an inlet-air filter screen.

The sterilization device includes a plasma device or an electrostatic negative oxygen ion device.

The plasma device includes at least one group of plasma generators.

Each of the at least one group of plasma generators includes an air ionization discharging electrode and a high-voltage power plug. The plasma generator is arranged at at least one inlet-air filter screen, and the high-voltage power plug is connected with the plasma generator.

Alternatively, the electrostatic negative oxygen ion device includes at least one group of negative oxygen ion generators, an air corona discharging electrode and a high-voltage power plug. Each of the at least one group of negative oxygen ion generators is arranged at the at least one inlet-air filter screen, and the high-voltage power plug is connected with the negative oxygen ion generator.

Preferably, the air conditioning system terminal includes a sterilization device and an inlet-air filter screen.

The sterilization device includes an ultraviolet device.

The ultraviolet device includes at least one ultraviolet lamp tube and a power plug for an ultraviolet lamp tube.

The ultraviolet lamp tube is arranged at at least one inlet-air filter screen, and the power plug for the ultraviolet lamp tube is connected with the ultraviolet lamp tube.

Preferably, the sterilization air conditioner includes an air conditioner indoor unit and a sterilization device.

The sterilization device includes a plasma device.

The plasma device at least includes a plasma generator. The plasma generator includes an air ionization discharging electrode and a high-voltage power plug. The plasma generator is arranged inside or outside at least one air inlet of the indoor cabinet of the air conditioner, and the high-voltage power plug is connected with the plasma generator.

Alternatively, the electrostatic negative oxygen ion device includes at least one group of negative oxygen ion generators, an air corona discharging electrode and a high-voltage power plug. Each of the at least one group of negative oxygen ion generators is arranged inside or outside at least one air inlet of the indoor cabinet of the air conditioner, and the high-voltage power plug is connected with the negative oxygen ion generator.

Preferably, the sterilization air conditioner includes an air conditioner indoor unit and a sterilization device.

The sterilization device includes at least one ultraviolet device, and the ultraviolet device at least includes an ultraviolet lamp tube and a power plug for the ultraviolet lamp tube. The ultraviolet lamp tube is arranged inside or outside the at least one air inlet of the indoor cabinet of the air conditioner or between the at least one air inlet of the indoor cabinet of the air conditioner and the surface air cooler of the air conditioner, and the power plug for the ultraviolet lamp tube is connected with the ultraviolet lamp tube.

Preferably, the sterilization air conditioner includes a fan-coil air conditioner, a sterilization device and a central air-conditioning return air inlet.

The sterilization device includes a plasma device. The plasma device includes a plasma generator. The plasma generator includes an air ionization discharging electrode and a high-voltage power plug. The plasma generator is arranged inside and/or outside the fan-coil air inlet, or the plasma generator is arranged between the fan-coil air inlet and the central air-conditioning return air inlet. The high-voltage power plug is connected with the plasma generator.

The sterilization device further includes the electrostatic negative oxygen ion device. The electrostatic negative oxygen ion device includes at least one group of negative oxygen ion generators, an air corona discharging electrode and a high-voltage power plug. Each of the at least one group of negative oxygen ion generators is arranged inside and/or outside the fan-coil air inlet, or the negative oxygen ion generators are arranged between the fan-coil air inlet and the central air-conditioning return air inlet. The high-voltage power plug is connected with the negative oxygen ion generator.

Preferably, the sterilization air conditioner includes a fan-coil air conditioner, a sterilization device and a central air-conditioning return air inlet.

The sterilization device includes an ultraviolet device.

The ultraviolet device includes an ultraviolet lamp tube and a power plug for the ultraviolet lamp tube. The ultraviolet lamp tube is arranged inside and/or outside the fan-coil air inlet, or between the fan-coil air inlet and the central air-conditioning return air inlet, and the power plug for the ultraviolet lamp tube is connected with the ultraviolet lamp tube.

The disclosure provides following beneficial effects.

1. The disinfectant is a globally recognized sterilization chemical reagent. Compared with other disinfection methods, it has advantages of low price, convenient use, simple operation, thorough sterilization and little toxic and side effects on human bodies, especially its low-temperature sterilization is more suitable for systematic requirements.

2. The disclosure can not only be applied to household and commercial split air conditioners and central air-conditioning fan-coil air conditioning systems that have been installed and used, but also can be used by factories to manufacture sterilization air conditioning products.

3. In the disclosure, sterilization operations can be carried out all year round: sterilization and refrigeration air conditioning operations can be realized using the device according to this disclosure when a refrigeration air conditioner is operated in summer. Sterilization and heating operations can be obtained by cooperating with the device according to this disclosure when heating is made in winter; and in spring and autumn, using a ventilation mode of the air conditioner, aseptic purifier devices in homes, guesthouses, hotels and office buildings can be created, and a sterile living and working environment can be created.

4. With spraying, atomizing and drenching of the disinfectant on circulating air of the air conditioner, most of pm2.5 particles in the air is washed by the disinfectant and into the disinfectant to be removed, and the circulating air is washed, filtered and purified. With sterilization effect of the disinfectant, dual functions of air purifier and sterilization can be realized. For existing homes, guesthouses, hotels and office buildings equipped with split air conditioners and fan-coil air conditioners, the split air conditioners and fan-coil air conditioners can be upgraded to sterilization air conditioners and air purification after equipped with the device according to the disclosure, which is of great environmental protection, hygiene and social significance.

### BRIEF DESCRIPTION OF THE DRAWINGS

In order to explain specific implementations of the present disclosure or technical schemes in related art more clearly, drawings required in the description of the specific implementations or the related art will be briefly introduced below; obviously, the drawings in the following description are some implementations of the present disclosure, and other drawings can be obtained according to these drawings by those of ordinary skill in the art without paying creative labor.

FIG. 1 is a schematic diagram of a structural embodiment of a split wall-mounted sterilization air conditioner according to the present disclosure.

FIG. 2 is a schematic diagram of a structural embodiment of another split wall-mounted sterilization air conditioner according to the present disclosure.

FIG. 3 is a schematic diagram of a structural embodiment of another split wall-mounted sterilization air conditioner according to the present disclosure.

FIG. 4 is a schematic diagram of a structural embodiment of a split cabinet sterilization air conditioner according to the present disclosure.

FIG. 5 is a schematic diagram of a structural embodiment of another split cabinet sterilization air conditioner according to the present disclosure.

FIG. 6 is a schematic diagram of a structural embodiment of yet another split wall-mounted sterilization air conditioner according to the present disclosure.

FIG. 7 is a schematic diagram of a structural embodiment of still another split wall-mounted sterilization air conditioner according to the present disclosure.

FIG. 8 is a schematic diagram of a structural embodiment of a split ceiling-concealed sterilization air conditioner according to the present disclosure.

FIG. 9 is a schematic diagram of a structural embodiment of a structure for storing disinfectants by using a shell of an indoor wall-hanging machine according to the present disclosure.

FIG. 10 is a schematic diagram of a structural embodiment of a structure for storing disinfectants by using a shell of an indoor cabinet machine according to the present disclosure.

FIG. 11 is a schematic diagram of a structural embodiment of a central air-conditioning fan-coil sterilization air conditioner according to the present disclosure.

FIG. 12 is a schematic diagram of a structural embodiment of another central air-conditioning fan-coil sterilization air conditioner according to the present disclosure.

FIG. 13 is a schematic diagram of a structural embodiment of another split wall-mounted sterilization air conditioner according to the present disclosure.

FIG. 14 is a schematic diagram of a structural embodiment of yet another split wall-mounted sterilization air conditioner according to the present disclosure.

FIG. 15 is a schematic diagram of a structural embodiment of another split cabinet sterilization air conditioner according to the present disclosure.

FIG. 16 is a schematic diagram of a structural embodiment of yet another split cabinet sterilization air conditioner according to the present disclosure.

FIG. 17 is a schematic diagram of a structural embodiment of another central air-conditioning fan-coil sterilization air conditioner according to the present disclosure.

FIG. 18 is a schematic diagram of a structural embodiment of yet another central air-conditioning fan-coil sterilization air conditioner according to the present disclosure.

Reference Numbers: 1. Air Conditioner Indoor Unit, 2. Air Inlet, 3. Air Outlet, 4. Panel of Inlet-air Filter Screen of Air Conditioner, 5. Filter Screen of Wall-hanging Machine, 6. Air Inlet of Indoor Cabinet of Air Conditioner, 7. Disinfectant Dripping, Drenching, Sprinkling, Spraying or Atomizing or Plasma or Ultraviolet Sterilization Device, 8. Disinfectant Storage Tank, 9. Disinfectant, 10. Disinfectant Ejection Port, 11. Ultrasonic Disinfectant Atomizing Device, 12. Ultrasonic Generator, 13. Ultrasonic Electric Control Device, 14. Disinfectant Atomizing Spray Pipe, 15. Blocking Plates on Two Sides of Housing of Air Conditioner, 16. Piping Port of Indoor Cabinet of Air Conditioner, 17. Disinfectant Pump, 18. Nozzle Connecting Pipe of Disinfectant Pump, 19. Nozzle, 20. Filter Screen of Cabinet, 21. Disinfectant Atomizing and Blasting Pipe, 22. Disinfectant Atomizing and Jetting Port, 23. Dripping Device, 24. Plasma Generator or Negative Oxygen Ion Generator. 25, Air Ionization or Corona Discharging Electrode, 26, High-voltage Power Plug, 27, Ultraviolet Lamp Tube, 28, Power Plug of Ultraviolet Lamp Tube, 29, Fan-coil Air Conditioner, 30, Fan-coil Air Inlet, 31, Fan-coil Fan, 32, Central Air-conditioning Return Air Inlet, 33, Plasma or Negative Oxygen Ion Device, 34, Ultraviolet Device, 35, Filter Screen at Central Air-conditioning Return Air Inlet.

### DETAILED DESCRIPTION

In the following, technical schemes in the disclosure will be described clearly and completely in connection with the embodiments; obviously, the described embodiments are intended to be a part of the embodiment of the disclosure, but not all of them. On a basis of the embodiments in this disclosure, all other embodiments obtained by the ordinary skilled in the art without any creative effort are within the protection scope of this disclosure.

In description of the present disclosure, it should be understood that terms “center”, “longitudinal”, “transverse”, “length”, “width”, “thickness”, “upper”, “lower”, “front”, “black”, “left”, “right”, “vertical” and “horizontal”, “top”, “bottom”, “inside”, “outside” which indicate an orientation or positional relationship are based on an orientation or positional relationship shown in the drawings, and are merely for convenience of describing the present disclosure and simplifying the description, rather than indicating or implying that the device or element referred to must have a specific orientation, be configured and operated in a specific orientation, and thus cannot be understood as limitation on the present disclosure.

In addition, terms “first” and “second” are only configured for descriptive purposes and cannot be understood as indicating or implying a relative importance, or implicitly indicating a number of indicated technical features. Therefore, features defined with “first” and “second” may explicitly or implicitly include one or more of the features. In the description of the present disclosure, “a plurality” means two or more, unless otherwise specifically defined. In addition, the terms “installing”, “coupling” and “connecting” should be understood in a broad sense, for example, it can be “fixedly connecting”, or “detachably connecting” or “integrally connecting”, or it can be “mechanically connecting” or “electrically connecting”, or it can be “directly connecting” or “indirectly connecting through an intermediate medium”, or it can be “communicating within two elements”. For a person of ordinary skill in the art, specific meanings of the above terms in the present disclosure can be understood according to specific situations.

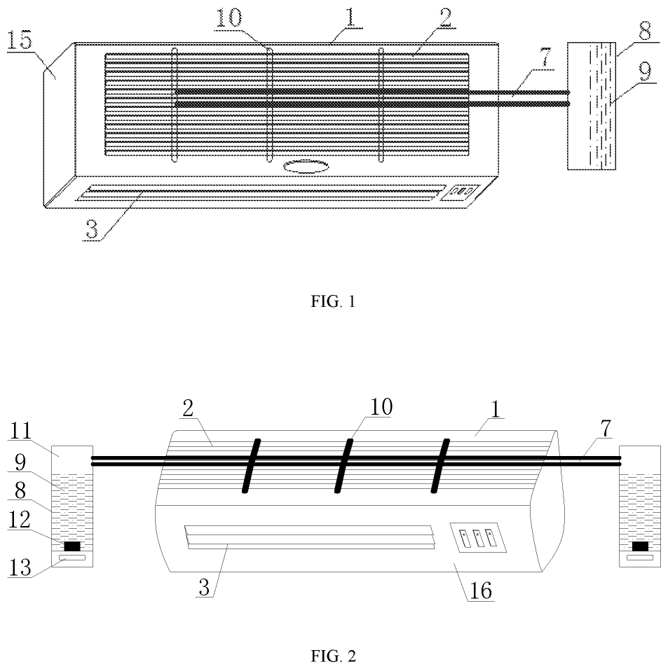

FIG. 1 is a schematic diagram of a structural embodiment of a split wall-mounted sterilization air conditioner according to the present disclosure.

n FIG. 1, an air conditioning system terminal is an air conditioner indoor unit 1, which includes an air inlet 2 and an air outlet 3. A sterilization device 7 includes a disinfectant storage tank 8, disinfectant 9 and a disinfectant ejection port arrangement 10. The disinfectant ejection port arrangement 10 includes at least one ejection port. The device is very suitable for existing installed split air conditioners, as a sterilization system for household ventilation air conditioners. The disinfectant 9 can be disinfectants such as hydrogen peroxide, hydrogen peroxide silver ion, chlorine dioxide, etc., especially the hydrogen peroxide silver ion disinfectant has less smell and excellent sterilization effect, which, with a proper proportion, can serve to realize real-time sterilization operation in presence of people. Compared with other sterilization devices, this device has advantages of small investment, quick effect and thorough sterilization, and is a practical sterilization device for families and businesses. The sterilization device 7 can be configured in applications outside the air conditioner to adapt to use for sterilization of the existing installed split air conditioner, and can also be directly designed and configured inside the air conditioner by an air conditioner manufacturer at a factory so as to produce sterilization air conditioner products. The disinfectant ejection port arrangement 10 is arranged outside the air inlet 2 of the air conditioner, which facilitates sterilization operations of a filter screen 5 of a wall-hanging machine. The disinfectant ejection port arrangement 10 is arranged in the air inlet 2 of the air conditioner, which facilitates the factory to produce sterilization air conditioner products. In particular, it is arranged between the surface air cooler and the filter screen 5 of the wall-hanging machine, which facilitates better sterilization effect for the surface air cooler and the filter screen 5 of the wall-hanging machine in operation at the same time.

FIG. 2 is a schematic diagram of a structural embodiment of another split wall-mounted sterilization air conditioner according to the present disclosure.

In FIG. 2, an air conditioning system terminal can be an air conditioner indoor unit 1, which includes an air inlet 2 and an air outlet 3. The sterilization device 7 includes at least one group of ultrasonic disinfectant atomizing devices 11. Each of at least one group of ultrasonic disinfectant atomizing devices 11 includes a disinfectant storage tank 8, disinfectant 9, a disinfectant ejection port arrangement 10, an ultrasonic generator 12 and an ultrasonic electric control device 13. In operating, ultrasonic high-frequency electric pulse current generated by the ultrasonic electric control device 13 is transmitted to the ultrasonic generator 12, and a piezoelectric ceramic sheet in the ultrasonic generator 12 generates ultrasonic vibration, and the disinfectant is excited and atomized by the ultrasonic vibration until disinfectant particles overflow and are transported to the disinfectant ejection port arrangement 10 through a pipeline, sprayed to the air inlet 2 by the disinfectant ejection port arrangement 10, and sucked by an operating air conditioner. Firstly, bacteria and viruses in the filter screen are inactivated by the filter screen 5 of the indoor wall-hanging machine of the air conditioner. When wind containing the disinfectant particles passes the surface air cooler after passing through the filter screen 5 of the indoor machine of the air conditioner, bacteria and viruses remaining in the surface air cooler are inactivated, and at the same time, air containing the disinfectant particles serve to inactivate viruses and bacteria remaining in space and parts in an air conditioner cavity, and finally sterilized air containing the disinfectant is blown into a room from the air outlet 3 to sterilize the room. However, disinfectant mist entering the air conditioner from the air inlet 2 is not only used to sterilize the filter screen 5 of the indoor machine of the air conditioner and the surface air cooler behind the filter screen 5 of the indoor machine of the air conditioner, because disinfectant mist air entering the air conditioner from the air inlet 2 is pervasive and basically sterilizes all inner cavities of the air conditioner. In summer, sterilization operations are made with refrigeration of the air conditioner. In winter, sterilization is made combined with heating. In spring and autumn, the air conditioner is set in a ventilation operation mode, and room ventilation and sterilization operations can be carried out. If a primary filter of the filter screen 5 of the indoor machine of the air conditioner is changed to be a medium efficient or sub-high efficient filter, not only pm2.5 particles can be eliminated but also sterilization can be made, with functions far exceeding that of an air purifier, realizing all-in-one applications. The ultrasonic disinfectant atomizing device 11 or the disinfectant storage tank 8 is made of transparent materials, so that change in disinfectant level can be clearly seen, and the disinfectant can be added in time. In FIG. 2, there are two ultrasonic disinfectant atomizing devices 11 which are arranged flat and close to the blocking plates on two sides of the air conditioner, which function in balancing left and right and increasing disinfectant capacity, thus prolonging sterilization duration and reducing times of adding disinfectant. The disinfectant is atomized or sprayed in inlet air of the air conditioner, which is equivalent to a process of washing air entering the air conditioner with disinfectant liquid, and pm2.5 particles in the air falls into the disinfectant mist and is purified, which is actually equivalent to a process of washing and purifying the air with water. Therefore, the sterilization air conditioner not only realizes the sterilization operation of the air conditioner, but also obtains the function of an air purifier.

The disinfectant 9 not only includes hydrogen peroxide or hydrogen peroxide silver ion or chlorine dioxide or formaldehyde or glutaraldehyde or ethylene oxide or peracetic acid or chlorine or copper sulfate or ethanol or isopropanol or n-propanol disinfectant, but also can be changed to recognized brands and customary types according to standards for using disinfectants in different countries. The sterilization device according to this disclosure supports sterilization applications compatible with any kind of air disinfectants.

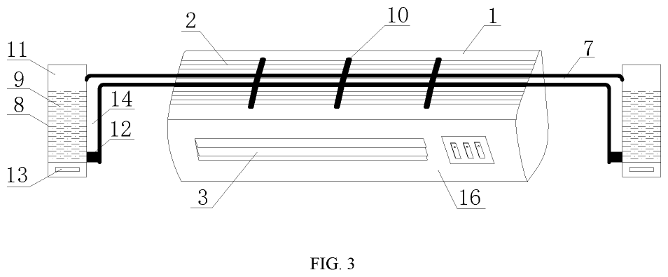

FIG. 3 is a schematic diagram of a structural embodiment of another split wall-mounted sterilization air conditioner according to the present disclosure.

In FIG. 3, other parts are exactly the same except that the ultrasonic generator 12 is arranged outside the disinfectant 9. The ultrasonic generator 12 is arranged outside the disinfectant 9, which facilitates atomization effect of the disinfectant to a certain degree, and other working principles are completely the same except the atomization effect is not influenced by a level or pressure of the disinfectant, and are not repeatedly described here again.

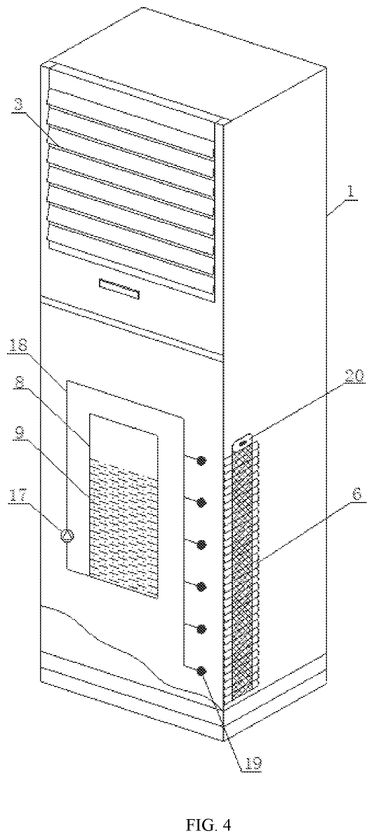

FIG. 4 is a schematic diagram of a structural embodiment of a split cabinet sterilization air conditioner according to the present disclosure.

In FIG. 4, an air conditioning system terminal includes an air conditioner indoor unit 1. The air conditioner indoor unit 1 includes an air outlet 3 and an air inlet 6 of an indoor cabinet of the air conditioner. The sterilization device 7 includes a disinfectant storage tank 8, disinfectant 9, a disinfectant pump 17, a nozzle connecting pipe 18 of the disinfectant pump and a nozzle 19. A disinfectant spray sterilization device is composed of a filter screen 20 of the indoor cabinet of the air conditioner. The nozzle 19 is arranged inside or outside the air inlet 6 of the indoor cabinet of the air conditioner. The disinfectant storage tank 8, the disinfectant 9 and the disinfectant pump 17 are arranged inside the cabinet. If the nozzle 19 is also arranged inside the air inlet 6 of the indoor cabinet of the air conditioner, it is convenient for manufacturers to manufacture sterilization air conditioner products. The nozzle 19 is arranged between the filter screen 20 of the indoor cabinet of the air conditioner and a fan, or at least one nozzle 19 is arranged at a side of filter screen 20 of the indoor cabinet of the air conditioner, for a case where the filter screen 20 of the indoor cabinet of the air conditioner is sterilized and/or the at least one nozzle 19 is arranged at a side of a fan of the indoor cabinet of the air conditioner, and at the same time for a case where the filter screen 20 of the indoor cabinet of the air conditioner is sterilized and the disinfectant mist is sucked in by the fan for sterilization of an inner structure of the fan, the surface air cooler and an internal cavity of the cabinet as well as components. When sterilization is made with the nozzle 19 being arranged inside the air inlet 6 of the indoor cabinet of the air conditioner, the disinfectant pump 17 is operated, and the disinfectant is pumped to the nozzle through the disinfectant pump and input to the nozzle 19 through the connecting pipe 18, and the mist is ejected from the nozzle 19. When the nozzle 19 is arranged inside the air inlet 6 of the indoor cabinet of the air conditioner, the disinfectant mist is uniformly sprayed on an inner side of the filter screen 20 of the indoor cabinet of the air conditioner for sterilization, and at the same time, the disinfectant mist is sucked into the surface air cooler and to the internal cavity of the cabinet and surfaces of the components for sterilization through a suction force from the fan. When the nozzle 19 is arranged outside the air inlet 6 of the indoor cabinet of the air conditioner, the connecting pipe 18 is led out through a piping port 16 of the indoor cabinet of the air conditioner and connected with the nozzle 19. The nozzle 19 is arranged and mounted outside the air inlet 6 of the indoor cabinet of the air conditioner, and the disinfectant mist ejected by the nozzle 19 is sucked by the air conditioner fan. The disinfectant mist serves to sterilize from the outside of the air inlet 6 of the indoor cabinet of the air conditioner until it passes through the air conditioner indoor cabinet filter screen 20 and enters the surface air cooler, interior of the cabinet and the components for overall sterilization.

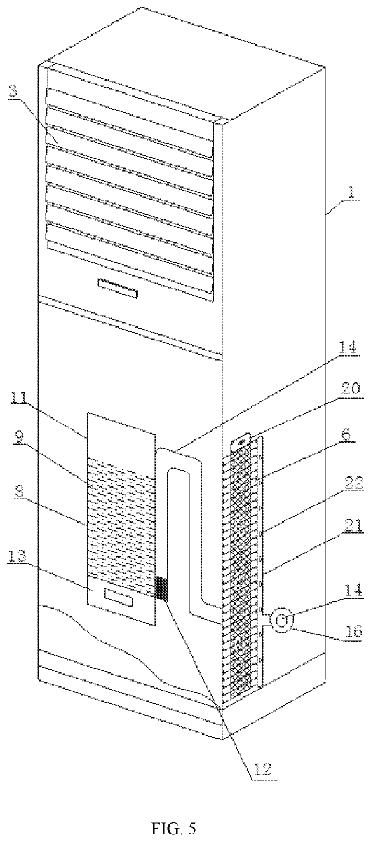

FIG. 5 is a schematic diagram of a structural embodiment of another split cabinet sterilization air conditioner according to the present disclosure. FIG. 5 is completely the same as FIG. 4 except the sterilization device 7. For the sterilization device 7 in FIG. 5, an ultrasonic disinfectant atomizing sterilization device is composed of a disinfectant storage tank 8, disinfectant 9, an ultrasonic disinfectant atomizing device 11, an ultrasonic generator 12, an ultrasonic electric control device 13, a disinfectant atomizing spray pipe 14, a disinfectant atomizing and blasting pipe 21 and a disinfectant atomizing and jetting port 22. Ultrasonic generated by the ultrasonic generator 12 excites to eject the disinfectant mist. The mist led out from a cabinet of an indoor unit 1 of the air conditioner through the disinfectant atomizing spray pipe 14 and the piping port 16 of the indoor cabinet of the air conditioner and then delivered through the connected disinfectant atomizing and blasting pipe 21 is ejected through the disinfectant atomizing and jetting port 22 and sucked by the fan of the operating air conditioner. The sucked disinfectant mist enters the surface air cooler for sterilization through the filter screen 20 of the indoor cabinet of the air conditioner, and sterile air is blown to an air-conditioned room from the air outlet 3, thus realizing the sterilization operation of the air conditioner. Other working processes are exactly the same as those in FIG. 4, which will not be repeatedly described here again. The connecting pipe 18 in FIG. 4 and the disinfectant atomizing spray pipe 14 in FIG. 5 are both led out through the piping port 16 of the indoor cabinet of the air conditioner, which makes the piping very easy. For aesthetics in factory production, the piping and positions and a connection mode of the nozzle 19 and the disinfectant atomizing and jetting port 22 can also be considered in a unified way.

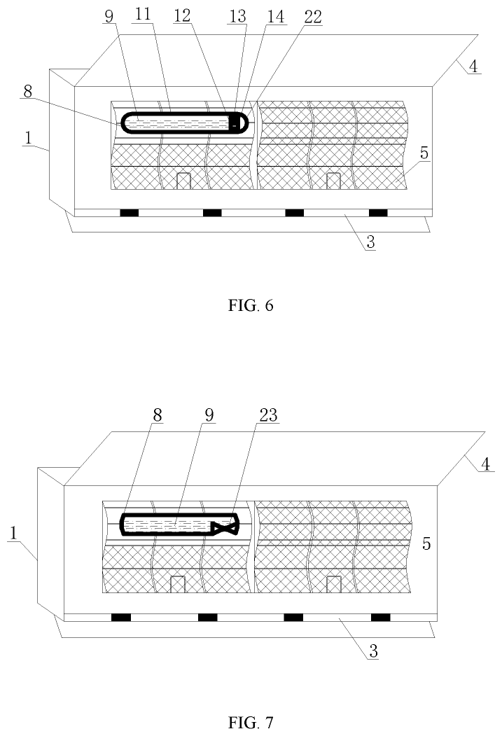

FIG. 6 is a schematic diagram of a structural embodiment of yet another split wall-mounted sterilization air conditioner according to the present disclosure. In FIG. 6, an ultrasonic disinfectant atomizing sterilization device is composed of a disinfectant storage tank 8, disinfectant 9, an ultrasonic disinfectant atomizing device 11, an ultrasonic generator 12, an ultrasonic electric control device 13, a disinfectant atomizing spray pipe 14 and a disinfectant atomizing and jetting port 22, which can be arranged on a bone frame of the filter screen 5 of the indoor unit of the air conditioner and can be freely disassembled and can serve to add disinfectant. The sterilization device can be arranged on one filter screen 5 of the indoor unit of the air conditioner or on two filter screens 5 of the indoor unit of the air conditioner. An ultrasonic atomization process is the same as that in FIG. 3, which will not be repeatedly described again. Since the ultrasonic disinfectant atomizing device 11 is arranged inside the air conditioner, a liquid level display window can be provided at a panel of the ultrasonic disinfectant atomizing device 11, and the liquid level can be directly seen from a panel 4 of an inlet-air filter screen of the air conditioner. Alternatively, an electronic liquid level control and liquid level alarm device can be provided to prompt to add the disinfectant. During the sterilization operation, the disinfectant mist generated by the ultrasonic generator 12 is ejected through the disinfectant atomizing and jetting port 22, and at least one disinfectant atomizing and jetting port 22 is configured to spray to the filter screen 5 of the indoor unit of the air conditioner, so that at least one or two filter screens 5 of the indoor unit of the air conditioner are sprayed with the disinfectant mist for sterilization. The at least one disinfectant atomizing and jetting port 22 is configured to spray to the surface air cooler and an interior and components of the air conditioner for sterilization.

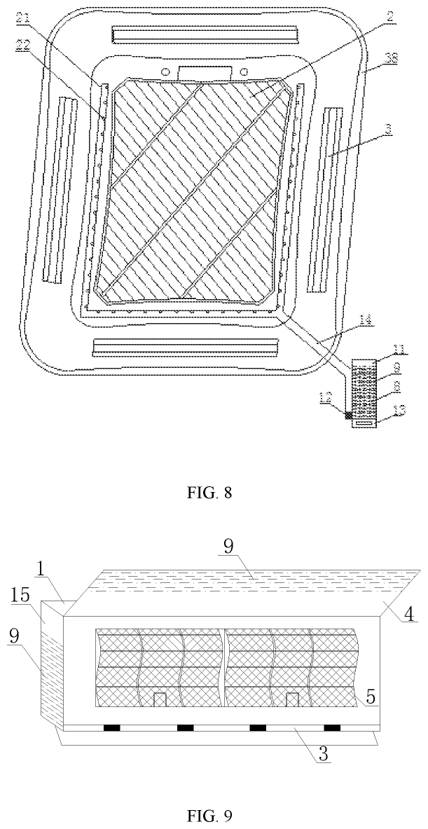

FIG. 7 is a schematic diagram of a structural embodiment of still another split wall-mounted sterilization air conditioner according to the present disclosure. In FIG. 7, a disinfectant dripping sterilization device is composed of a disinfectant storage tank 8, disinfectant 9 and a dripping device 23. As is the same as FIG. 6, the disinfectant dripping sterilization device is arranged on the bone frame of the filter screen 5 of the indoor unit of the air conditioner, can be freely disassembled and can serve to add disinfectant. The sterilization device can be arranged on one filter screen 5 of the indoor unit of the air conditioner or on two filter screens 5 of the indoor unit of the air conditioner. The dripping device 23 is communicated with the disinfectant 9. The dripping device 23 is composed of a micro valve, a medical infusion regulating valve or a device that can control a liquid flow. During sterilization, the disinfectant is slowly dripped out by the dripping device 23 and on the filter screen 5 of the indoor unit of the air conditioner, and a dripping speed is adjusted to just make the filter screen 5 of the indoor unit of the air conditioner be evenly covered with the disinfectant, but not so saturated that the disinfectant drips from the filter screen 5 of the indoor unit of the air conditioner; and suitability can be adjusted so that sterilization can be carried out and the disinfectant can be saved. Liquid level display and alarm measures are the same as those in FIG. 7, which will not be repeatedly described here again.

FIG. 8 is a schematic diagram of a structural embodiment of a split ceiling-concealed sterilization air conditioner according to the present disclosure. A sterilization device of FIG. 8 is basically the same as those of FIGS. 3 and 5, except that the air conditioner is a ceiling-concealed indoor unit 38. Due to the ceiling-concealed structure, the disinfectant storage tank 8, the disinfectant 9, the ultrasonic disinfectant atomizing device 11, the ultrasonic generator 12, the ultrasonic electric control device 13 and the disinfectant atomizing spray pipe 14 can all be arranged in a ceiling for aesthetics. The disinfectant atomizing spray pipe 14 is led out from a ceiling decorative board and connected with the disinfectant atomizing and blasting pipe 21. In operation, the atomized disinfectant mist is ejected through the disinfectant atomizing and jetting port 22 and sucked in through the air inlet 2, and interiors of the filter screen, the surface air cooler, the components and the cavity of a ceiling-concealed machine can be completely sterilized. The ultrasonic disinfectant atomizing device 11 is arranged in the ceiling-concealed indoor unit, which facilitates manufacturing and selling of sterilization air conditioners. The disinfectant atomizing spray pipe 14 can be arranged outside or inside the air inlet 2, but it is more ideal to be arranged outside the air inlet.

FIG. 9 is a schematic diagram of a structural embodiment of a structure for storing disinfectants by using a shell of an indoor wall-hanging machine according to the present disclosure. In FIG. 9, the panel 4 of an inlet-air filter screen of the air conditioner and the blocking plates 15 on both sides of the shell of the air conditioner are used and transformed into the blocking plates and panel with hollow structures, and hollow cavity space capacity is used as the disinfectant storage tank 8 and to store the disinfectant 9. Then, the ultrasonic generator 12 and the ultrasonic electric control device 13 are arranged and separately mounted at proper positions on the panel 4 of an inlet-air filter screen of the air conditioner or the blocking plates 15 on both sides of the shell of the air conditioner, so that they are arranged and mounted in the panel 4 of an inlet-air filter screen of the air conditioner or the blocking plates 15 on both sides of the shell of the air conditioner and at side of interior of the air conditioner, so as to be communicated with the disinfectant 9 to form the sterilization air conditioner and to spray the disinfectant mist liquid on the whole interior of the air conditioner. A liquid level display device and a disinfectant filling port are arranged at appropriate positions outside the panel 4 of an inlet-air filter screen of the air conditioner or the blocking plates 15 on both sides of the shell of the air conditioner, which not only saves space, but also can be manufactured into an excellent sterilization air conditioner product with an original air conditioner structure unchanged.

In FIG. 9, a hollow structure also can be made by using a rear shell plate facing a wall and a bottom plate, and a hollow cavity is used as the disinfectant storage tank 8 and to store the disinfectant 9.

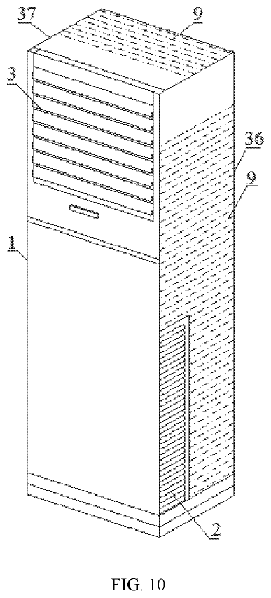

FIG. 10 is a schematic diagram of a structural embodiment of a structure for storing disinfectants by using a housing of an indoor cabinet machine according to the present disclosure. FIG. 10 is basically the same as FIG. 9, with difference in that the disinfectant storage tank 8 is manufactured by using a shell of an indoor cabinet machine to store the disinfectant 9. In FIG. 10, one or both sides of a cabinet decoration plate 36 are made into a hollow structure, and a cavity is made to replace the disinfectant storage tank 8 to store the disinfectant 9. A cabinet top panel 37 is made into a hollow structure, and a cavity is made to replace the disinfectant storage tank 8 to store the disinfectant 9. Of course, a back plate and a bottom plate can also be made into a hollow structure, and a cavity is made to replace the disinfectant storage tank 8 to store the disinfectant 9. Due to a large space of the cabinet, it is very convenient to install the disinfectant sterilization device, for which a specific implementation has been introduced before and will not be repeatedly described here again.

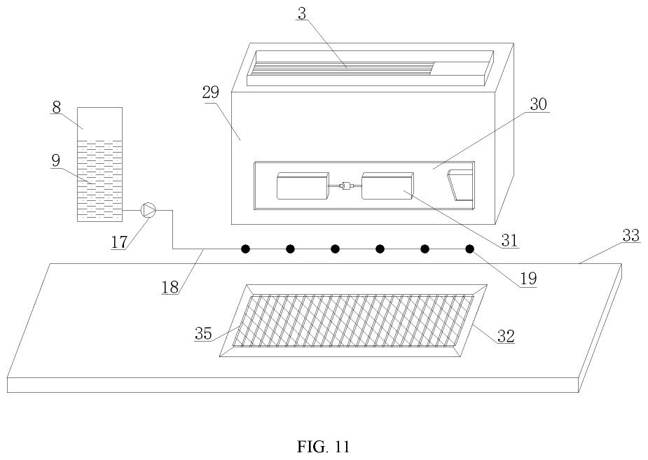

FIG. 11 is a schematic diagram of a structural embodiment of a central air-conditioning fan-coil sterilization air conditioner according to the present disclosure. FIG. 11 is a schematic diagram of an embodiment of a disinfectant spray sterilization device provided on a central air-conditioning fan-coil air conditioner according to the present disclosure. FIG. 11 is a device for sterilizing a fan-coil air conditioner of a central air conditioning system. The sterilization device 7 includes a disinfectant storage tank 8, disinfectant 9, a disinfectant pump 17, a nozzle connecting pipe 18 of the disinfectant pump and a nozzle 19. The central air-conditioning fan-coil air conditioner includes a fan-coil air conditioner 29, a fan-coil air inlet 30, a fan-coil fan 31, a central air-conditioning return air inlet 32 and a filter screen 35 at the central air-conditioning return air inlet. The central air-conditioning return air inlet 32 is mounted on a ceiling decorative board below the fan-coil air inlet 30. Generally, the filter screen 35 at the central air-conditioning return air inlet is arranged on the central air-conditioning return air inlet 32, and the filter screen 35 can be disassembled and cleaned. FIG. 11 shows that a disinfectant spray sterilization device is arranged in the ceiling, and the nozzle 19 is arranged between the filter screen 35 at the central air-conditioning return air inlet and the fan-coil air inlet 30, and spray is directed to the filter screen 35 at the central air-conditioning return air inlet. In operation, the disinfectant pump 17 is operated, and the disinfectant spray enters the nozzle 19 through the nozzle connecting pipe 18 of the disinfectant pump, and the disinfectant mist is uniformly sprayed from the nozzle 19 to the filter screen 35 at the central air-conditioning return air inlet for sterilization, and the mist is also sucked by the operated coil fan to sterilize the surface air cooler. At least one or more nozzles 19 are provided to achieve uniform disinfectant mist. The nozzle 19 is arranged between the filter screen 35 at the central air-conditioning return air inlet and the fan-coil air inlet 30, and there is at least one filter screen 35 at the central air-conditioning return air inlet and at least one nozzle 19 sprays in a direction of the fan-coil air inlet 30 at the same time for joint spray sterilization. The above sterilization device can also be provided in the fan coil by the factory and sold as a sterilization fan coil product.

For existing installed fan coils, the disinfectant storage tank 8, the disinfectant 9 and the disinfectant pump 17 can be arranged in the ceiling and are led out of the ceiling decorative plate through the nozzle connecting pipe 18 of the disinfectant pump so as to be connected with the nozzle 19. The nozzle 19 is arranged on the outside of the central air-conditioning return air inlet 32, and the spray is directed to the central air-conditioning return air inlet 32, and the mist can be sprayed to the central air-conditioning return air inlet 32 for sterilization. Of course, if it is inconvenient to install the ceiling, the disinfectant storage tank 8, the disinfectant 9 and the disinfectant pump 17 can also be arranged outside the ceiling.

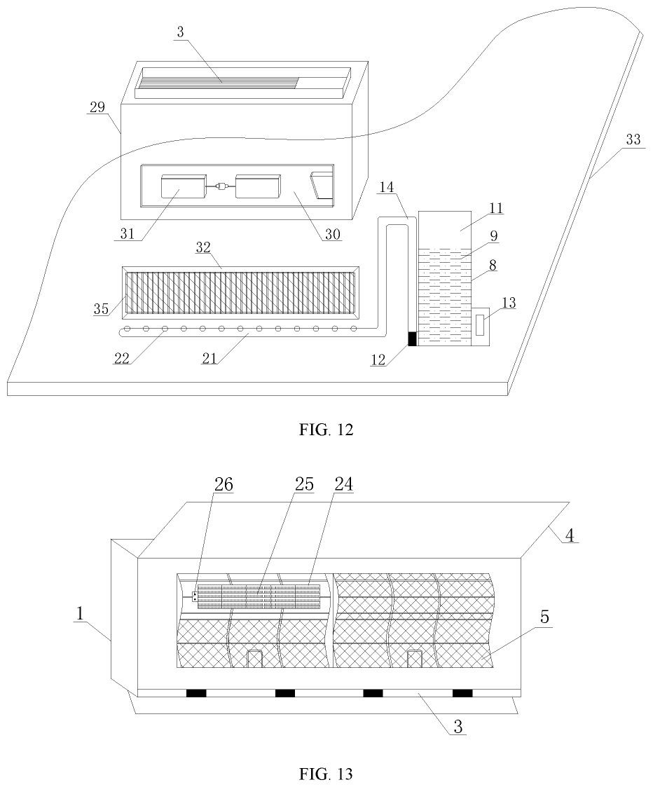

FIG. 12 is a schematic diagram of a structural embodiment of another central air-conditioning fan-coil sterilization air conditioner according to the present disclosure. FIG. 12 is different from FIG. 11 in that an ultrasonic disinfectant atomizing sterilization device 7 is composed of a disinfectant storage tank 8, disinfectant 9, an ultrasonic disinfectant atomizing device 11, an ultrasonic generator 12, an ultrasonic electric control device 13, a disinfectant atomizing spray pipe 14, a disinfectant atomizing and blasting pipe 21 and a disinfectant atomizing and jetting port 22, and its working principle is exactly the same as that of FIG. 5, which will not be repeatedly described here again. The ultrasonic disinfectant atomizing device 11 is arranged in the ceiling, the disinfectant atomizing and blasting pipe 21 is arranged near the central air-conditioning return air inlet 32 outside the ceiling, and the disinfectant atomizing and jetting port 22 is directed to the central air-conditioning return air inlet 32. In operation, the disinfectant mist ejected by the ultrasonic generator 12 is sprayed from the disinfectant atomizing and jetting port 22 through the disinfectant atomizing spray pipe 14 and the disinfectant atomizing and blasting pipe 21, and is sucked by the fan of the operating fan coil, and then passes and serves to sterilize the filter screen 35 at the central air-conditioning return air inlet and then enters the fan coil and the surface air cooler for sterilization. Alternatively, the disinfectant atomizing and blasting pipe 21 can be arranged near the central air-conditioning return air inlet 32 in the ceiling, and the disinfectant mist can be sprayed into the filter screen 35 at the central air-conditioning return air inlet for sterilization.

FIG. 13 is a schematic diagram of a structural embodiment of another split wall-mounted sterilization air conditioner according to the present disclosure. FIG. 13 is a schematic diagram of an embodiment of a plasma sterilization device arranged inside an indoor wall-hanging machine of a split air conditioner according to the present disclosure. It is completely the same as that in FIGS. 6 and 7 except for a sterilization mode. In FIG. 13, a plasma sterilization device is composed of a plasma device 33, a plasma generator 24, an air corona discharging electrode 25 and a high-voltage power plug 26. High-voltage and ultra-high-voltage power is supplied by the plasma device 33 and output to the plasma generator 24 through the high-voltage power plug 26, and are discharged via the air ionization discharging electrode 25 to ionize the air, so that the ionized gaseous substance consisting of atoms in air molecules for which some electrons are deprived and positive and negative ions generated by ionizing the radicals is called a plasma. In operating, the plasma electrostatic field decomposes and destroys negatively charged bacteria, and bacteria and viruses that lose electrons may lose their activity and absorb dust in the air, thus realizing air sterilization and dust removal.

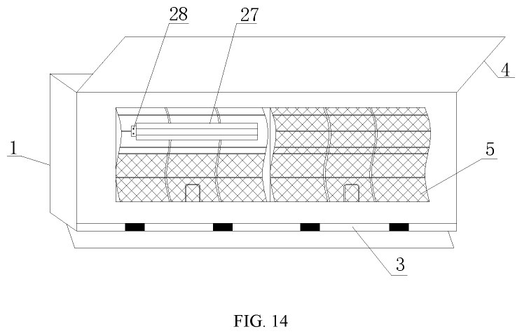

FIG. 14 is a schematic diagram of a structural embodiment of yet another split wall-mounted sterilization air conditioner according to the present disclosure. In FIG. 14, an ultraviolet sterilization device consisting of an ultraviolet lamp tube 27, a power plug 28 for the ultraviolet lamp tube and an ultraviolet device 34 is used for sterilization, which is completely the same as that in FIGS. 6, 7 and 13 except for a sterilization mode. Ultraviolet sterilization is to destroy a molecular structure of deoxyribonucleic acid (DNA) or ribonucleic acid (RNA) in microbial cells by using ultraviolet rays with appropriate wavelengths, resulting in death of growing cells or regenerative cells, thus achieving sterilization effect. The ultraviolet rays involves radiation disinfection, and therefore, the ultraviolet rays cannot diffract, so a surface of an object directly irradiated can be sterilized, and spaces and objects that cannot be irradiated cannot be sterilized. The ultraviolet lamp tube 27 is arranged on one filter screen 5 of the indoor unit of the air conditioner or on two filter screens 5 of the indoor unit of the air conditioner, or between the filter screen 5 of the indoor unit of the air conditioner and the surface air cooler of the air conditioner. In operating, high-frequency electromagnetic waves generated by the ultraviolet device 34 are transmitted to the ultraviolet lamp tube 27 through the power plug 28 for the ultraviolet lamp tube to generate ultraviolet rays, which are irradiated on the filter screen 5 of the indoor unit of the air conditioner for sterilization or directly irradiated on a surface of the filter screen 5 of the indoor unit of the air conditioner and a surface of the surface air cooler for multi-layer sterilization.

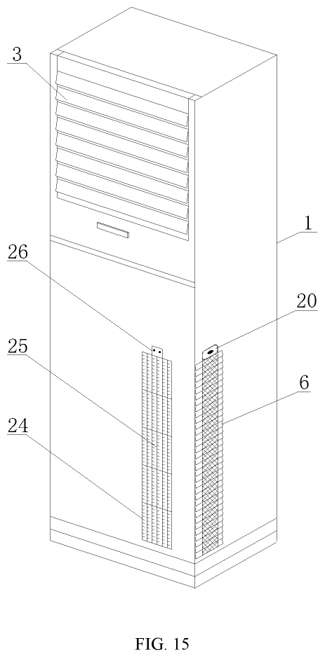

FIG. 15 is a schematic diagram of a structural embodiment of another split cabinet sterilization air conditioner according to the present disclosure. A sterilization device of FIG. 15 is the same as that of FIG. 13, except that the indoor unit of the air conditioner is different. The plasma generator 24 shown in FIG. 15 is arranged inside the air inlet 6 of the indoor cabinet of the air conditioner, mainly for safety. In operating, the plasma released by the plasma generator 24 serves to sterilize the filter screen 20 of a cabinet above the air inlet 6 of the indoor cabinet of the air conditioner, and at the same time, the plasma is sucked by the fan so as to sterilize cavities in the surface air cooler, the components and the cabinet.

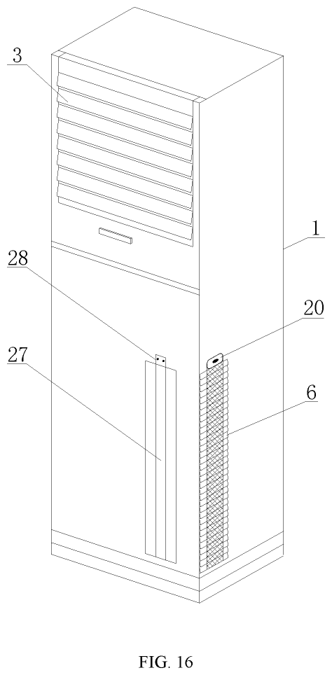

FIG. 16 is a schematic diagram of a structural embodiment of yet another split cabinet sterilization air conditioner according to the present disclosure. A sterilization device of FIG. 16 is the same as that of FIG. 14, except that the indoor unit of the air conditioner is different. The ultraviolet lamp tube 27 shown in FIG. 16 is arranged inside the air inlet 6 of the indoor cabinet of the air conditioner, mainly for safety. In operating, the ultraviolet rays released by the ultraviolet lamp tube 27 serves to sterilize the filter screen 20 of the cabinet above the air inlet 6 of the indoor cabinet of the air conditioner, and at the same time to sterilize the fan components and the cavity in the cabinet.

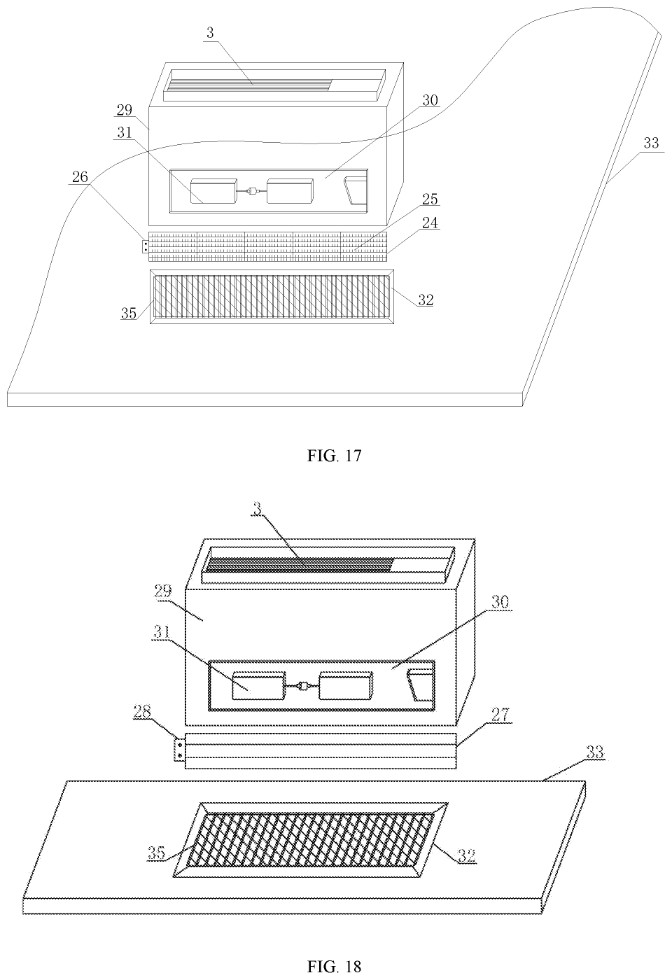

FIG. 17 is a schematic diagram of a structural embodiment of another central air-conditioning fan-coil sterilization air conditioner according to the present disclosure. FIG. 17 is the same as FIGS. 11 and 12 except that the sterilization device is different. The plasma generator 24 shown in FIG. 17 is arranged between the fan-coil air inlet 30 and the central air-conditioning return air inlet 32, mainly for safety. In operating, plasma released by the plasma generator 24 serves to sterilize the filter screen 35 at the central air-conditioning return air inlet arranged above the fan-coil air inlet 30, and at the same time, the plasma is sucked by the fan-coil air inlet 30 to sterilize cavities of the fan, the surface air cooler, the components and the fan coil.

FIG. 18 is a schematic diagram of a structural embodiment of yet another central air-conditioning fan-coil sterilization air conditioner according to the present disclosure. A sterilization device of FIG. 18 is the same as that of FIG. 14 except for a type of the air conditioner. The ultraviolet lamp tube 27 shown in FIG. 18 is arranged between the fan-coil air inlet 30 and the central air-conditioning return air inlet 32, mainly for safety. In operating, the ultraviolet rays released by the ultraviolet lamp tube 27 serves to sterilize the filter screen 35 at the central air-conditioning return air inlet arranged above the fan-coil air inlet 30, and at the same time, the ultraviolet rays irradiate and sterilize the cavities of the fan, the surface air cooler, the components and the fan coil.

Finally, it should be noted that above embodiments are only used to illustrate, but not to limit, technical schemes of the present disclosure. Although the disclosure has been described in detail with reference to foregoing embodiments, it should be understood by those skilled in the art that modifications still can be made to the technical schemes described in the foregoing embodiments or equivalent substitutions can be made to some or all of technical features thereof. These modifications and substitutions do not cause essence of the corresponding technical schemes to depart from scopes of technical schemes of various embodiments of the present disclosure.

What is claimed is:

1. A sterilization air conditioner, comprising an air conditioning system terminal and a sterilization device; wherein
the sterilization device is arranged on the air conditioning system terminal, and is configured for sterilizing air entering the air conditioning system terminal and the air conditioning system terminal.

2. The sterilization air conditioner according to claim 1, wherein the air conditioning system terminal comprises an indoor unit or a fan coil;
the indoor unit comprising a split-type wall-mounted air conditioning unit, a cabinet and a ceiling-concealed indoor unit; and
the fan coil comprising a surface mounted fan coil and a concealed fan coil.

3. The sterilization air conditioner according to claim 1, wherein the sterilization device comprises a physical sterilization device and/or a chemical sterilization device; wherein
the chemical sterilization device is configured to sterilize by releasing disinfectant by a dripping, drenching, sprinkling, spraying or atomizing device; and
the physical sterilization device is configured to sterilize by releasing plasma or ultraviolet rays or negative oxygen ions.

4. The sterilization air conditioner according to claim 1, wherein the disinfectant (9) comprises hydrogen peroxide or hydrogen peroxide silver ion or chlorine dioxide or formaldehyde or glutaraldehyde or ethylene oxide or peracetic acid or chlorine or hypochlorous acid or copper sulfate or ethanol or isopropanol or n-propanol disinfectant.

5. The sterilization air conditioner according to claim 1, wherein the sterilization device (7) comprises a disinfectant storage tank (8), disinfectant (9) and a disinfectant ejection port arrangement (10); wherein
the disinfectant (9) is stored in the disinfectant storage tank (8), the disinfectant ejection port arrangement (10) is communicated with the disinfectant (9), the disinfectant ejection port arrangement (10) is arranged inside or outside the air inlet (2) of the air conditioning system terminal (1), and the disinfectant ejection port arrangement (10) includes at least one ejection port; and
the sterilization device (7) is arranged outside the air conditioning system terminal (1) or inside the air conditioning system terminal (1).

6. The sterilization air conditioner according to claim 1, wherein the sterilization device (7) comprises at least one group of ultrasonic disinfectant atomizing devices (11); wherein
each of at least one group of ultrasonic disinfectant atomizing devices (11) comprises a disinfectant storage tank (8), disinfectant (9), an ultrasonic generator (12) and an ultrasonic electric control device (13); wherein
the ultrasonic generator (12) is immersed within the disinfectant (9), the ultrasonic disinfectant atomizing device (11) is communicated with the disinfectant ejection port arrangement (10), the ultrasonic electric control device (13) is connected with the ultrasonic generator (12), and the disinfectant ejection port arrangement (10) is arranged inside or outside the air inlet (2) of the air conditioning system terminal; and
the sterilization device (7) is arranged outside the air conditioning system terminal (1) or inside the air conditioning system terminal (1).

7. The sterilization air conditioner according to claim 1, wherein the sterilization device (7) comprises at least one group of ultrasonic disinfectant atomizing devices (11); wherein
each of the at least one group of ultrasonic disinfectant atomizing devices (11) comprises a storage tank (8), disinfectant (9), an ultrasonic generator (12), an ultrasonic electric control device (13) and a disinfectant atomizing spray pipe (14); wherein
the ultrasonic generator (12) is arranged outside the disinfectant (9) and the ultrasonic generator (12) is communicated with the disinfectant (9); the disinfectant atomizing spray pipe (14) is communicated with the disinfectant ejection port arrangement (10), the ultrasonic electric control device (13) is connected with the ultrasonic generator (12), and the disinfectant ejection port arrangement (10) is arranged inside or outside the air inlet of the air conditioning system terminal (2); and
the sterilization device (7) is arranged outside the air conditioning system terminal (1) or inside the air conditioning system terminal (1).

8. The sterilization air conditioner according to claim 1, wherein the sterilization device (7) comprises a disinfectant storage tank (8), disinfectant (9), a disinfectant pump (17) and at least one nozzle (19); wherein
the disinfectant pump (17) is communicated with the disinfectant (9), the nozzle (19) is connected with the disinfectant pump (17), and the nozzle (19) is arranged inside or outside an air inlet (6) of an indoor cabinet of the air conditioner at the air conditioning system terminal; and
the sterilization device (7) is arranged outside the air conditioning system terminal (1) or inside the air conditioning system terminal (1).

9. The sterilization air conditioner according to claim 1, wherein the sterilization device (7) comprises an ultrasonic disinfectant atomizing device (11), a disinfectant atomizing and blasting pipe (21) and at least one disinfectant atomizing and jetting port (22); wherein
the disinfectant atomizing and blasting pipe (21) is communicated with the disinfectant atomizing spray pipe (14), the disinfectant atomizing and jetting port (22) is connected with the disinfectant atomizing and blasting pipe (21), and the disinfectant atomizing and jetting port (22) and the disinfectant atomizing and blasting pipe (21) are arranged inside or outside the air inlet (6) of the indoor cabinet of the air conditioner at the air conditioning system terminal; and
the sterilization device (7) is arranged outside the air conditioning system terminal (1) or inside the air conditioning system terminal (1).

10. The sterilization air conditioner according to claim 1, comprising a sterilization device (7), an air inlet (2) of the air conditioning system terminal and an inlet-air filter screen (5); wherein
the sterilization device (7) comprises a storage tank (8), disinfectant (9), at least one ultrasonic disinfectant atomizing device (11), an ultrasonic generator (12), an ultrasonic electric control device (13), a disinfectant atomizing spray pipe (14) and at least one disinfectant atomizing and jetting port (22); wherein
the ultrasonic generator (12) is communicated with the disinfectant (9), the ultrasonic generator (12) is connected with the ultrasonic electric control device (13), and the disinfectant atomizing spray pipe (14) is communicated with the disinfectant atomizing and jetting port (22);
the ultrasonic disinfectant atomizing device (11) is at least arranged on one inlet-air filter screen (5); and
the sterilization device (7) is arranged outside the air conditioning system terminal (1) or inside the air conditioning system terminal (1).

11. The sterilization air conditioner according to claim 1, wherein the sterilization device (7) comprises at least one dripping device (23), the at least one dripping device (23) comprising a disinfectant storage tank (8), a disinfectant (9) and a dripping device (23); wherein
the dripping device (23) is communicated with the disinfectant (9), and the dripping device (23) can be a valve or a medical drip control valve and is configured for dripping disinfectant liquid to at least one inlet-air filter screen (5); and
the sterilization device (7) is arranged outside the air conditioning system terminal (1) or inside the air conditioning system terminal (1).

12. The sterilizing air conditioner according to claim 1, wherein the sterilizing device (7) comprises an ultrasonic disinfectant atomizing device (11); wherein
the ultrasonic disinfectant atomizing device (11) comprises a storage tank (8), disinfectant (9), an ultrasonic generator (12), an ultrasonic electric control device (13), a disinfectant atomizing spray pipe (14), at least one disinfectant atomizing and blasting pipe (21) and at least one disinfectant atomizing and jetting port (22); wherein
the disinfectant atomizing and blasting pipe (21) is communicated with the disinfectant atomizing spray pipe (14), the disinfectant atomizing and jetting port (22) is connected with the disinfectant atomizing and blasting pipe (21), the disinfectant atomizing and jetting port (22) and the disinfectant atomizing and blasting pipe (21) are arranged inside or outside the air inlet (2) of the air conditioning system terminal, the ultrasonic generator (12) is arranged outside the disinfectant (9), the ultrasonic generator (12) is communicated with the disinfectant (9), and the ultrasonic electric control device (13) is connected with the ultrasonic generator (12); and
the sterilization device (7) is arranged outside the air conditioning system terminal (1) or inside the air conditioning system terminal (1).

13. The sterilization air conditioner according to claim 4, wherein a shell of the indoor unit and/or a panel of an inlet-air filter screen are of a structure with a cavity, and the shell with the cavity constitutes the storage tank and stores the disinfectant.

14. The sterilization air conditioner according to claim 1, comprising a fan-coil air conditioner (29), a fan-coil air inlet (30) and a return air inlet (32) of the fan-coil air conditioner; wherein
the sterilization device (7) comprises a disinfectant storage tank (8), disinfectant (9), a disinfectant pump (17) and at least one nozzle (19); wherein
the fan-coil air conditioner (29) is arranged in a ceiling of an air-conditioned room, the disinfectant storage tank (8) is arranged in or outside the ceiling of the air-conditioned room, and the return air inlet (32) of the fan-coil air conditioner is arranged on the ceiling of the air-conditioned room; and
the disinfectant pump (17) is connected with the disinfectant storage tank (8), the disinfectant pump (17) is communicated with the disinfectant (9), and the nozzle (19) is connected with the disinfectant pump (17), the nozzle (19) is arranged at an air inlet (30) of the fan-coil air conditioner (29) or at the return air inlet (32) of the fan-coil air conditioner, or inside or outside the air return outlet (3) of the fan-coil air conditioner.

15. The sterilization air conditioner according to claim 1, comprising a fan-coil air conditioner (29), a fan-coil air inlet (30) and a return air inlet (32) of the fan-coil air conditioner; wherein
the sterilization device (7) comprises an ultrasonic disinfectant atomizing device (11), a disinfectant atomizing and blasting pipe (21) and at least one disinfectant atomizing and jetting port (22); wherein
the ultrasonic disinfectant atomizing device (11) comprises a disinfectant storage tank (8), disinfectant (9), an ultrasonic generator (12), an ultrasonic electric control device (13) and a disinfectant atomizing spray pipe (14); wherein
the ultrasonic generator (12) is arranged outside the disinfectant (9) and is communicated with the disinfectant (9), the ultrasonic electric control device (13) is connected with the ultrasonic generator (12), the disinfectant atomizing and blasting pipe (21) is communicated with the disinfectant atomizing and jetting port (22), and the disinfectant atomizing and blasting pipe (21) is connected with the disinfectant atomizing spray pipe (14);
the fan-coil air conditioner (29) is arranged in a ceiling of an air-conditioned room, and the return air inlet (32) of the fan-coil air conditioner is arranged on the ceiling of the air-conditioned room; and
the disinfectant atomizing and blasting pipe (21) is arranged inside or outside the air inlet of the fan-coil air conditioner (29), or inside or outside the return air inlet (32) of the fan-coil air conditioner, the disinfectant atomizing and blasting pipe (21) is connected with the disinfectant atomizing spray pipe (14), the disinfectant atomizing and jetting port (22) is communicated with the disinfectant atomizing and blasting pipe (21), and the ultrasonic disinfectant atomizing device (11) is arranged inside or outside the ceiling of the air-conditioned room.

16. The sterilization air conditioner according to claim 1, wherein the air conditioning system terminal comprises a sterilization device (7) and an inlet-air filter screen (5); wherein
the sterilization device (7) comprises a plasma device or an electrostatic negative oxygen ion device (33);
the plasma device (33) comprises at least one group of plasma generators (24); wherein
each of the at least one group of plasma generators (24) comprises an air ionization discharging electrode (25) and a high-voltage power plug (26), the plasma generator (24) is arranged at at least one inlet-air filter screen (5), and the high-voltage power plug (26) is connected with the plasma generator (24); or
the electrostatic negative oxygen ion device (33) comprises at least one group of negative oxygen ion generators (24), an air corona discharging electrode (25) and a high-voltage power plug (26), each of the at least one group of negative oxygen ion generators (24) being arranged at the at least one inlet-air filter screen (5) and the high-voltage power plug (26) being connected with the negative oxygen ion generator (24).

17. The sterilization air conditioner according to claim 1, wherein the air conditioning system terminal comprises a sterilization device (7) and an inlet-air filter screen (5); wherein
the sterilization device (7) comprises an ultraviolet device (34); wherein
the ultraviolet device (34) comprising at least one ultraviolet lamp tube (27) and a power plug (28) for an ultraviolet lamp tube; and
the ultraviolet lamp tube (27) being arranged at at least one inlet-air filter screen (5), and the power plug (28) for the ultraviolet lamp tube being connected with the ultraviolet lamp tube (27).

18. The sterilization air conditioner according to claim 1, comprising an air conditioner indoor unit (1) and a sterilization device (7); wherein
the sterilization device (7) comprises a plasma device (33); wherein
the plasma device (33) comprises at least one plasma generator (24), the at least one plasma generator (24) comprising an air ionization discharging electrode (25) and a high-voltage pow plug (26), the plasma generator (24) being arranged inside or outside at least one air inlet (6) of the indoor cabinet of the air conditioner, and the high-voltage power plug (26) being connected with the plasma generator (24); or
the electrostatic negative oxygen ion device (33) comprises at least one group of negative oxygen ion generators (24), an air corona discharging electrode (25) and a high-voltage power plug (26), each of the at least one group of negative oxygen ion generators (24) being arranged inside or outside at least one air inlet (6) of the indoor cabinet of the air conditioner, and the high-voltage power plug (26) being connected with the negative oxygen ion generator (24).

19. The sterilization air conditioner according to claim 1, comprising an air conditioner indoor unit (1) and a sterilization device (7); wherein
the sterilization device (7) comprises at least one ultraviolet device (34), and the at least one ultraviolet device (34) at least comprising an ultraviolet lamp tube (27) and a power plug (28) for the ultraviolet lamp tube, the ultraviolet lamp tube (27) being arranged inside or outside the at least one air inlet (6) of the indoor cabinet of the air conditioner or between the at least one air inlet (6) of the indoor cabinet of the air conditioner and the surface air cooler of the air conditioner, the power plug (28) for the ultraviolet lamp tube being connected with the ultraviolet lamp tube (27).

20. The sterilization air conditioner according to claim 1, comprising a fan-coil air conditioner (29), a sterilization device (7) and a central air-conditioning return air inlet (32);
wherein the sterilization device (7) comprises a plasma device (33), the plasma device (33) comprising a plasma generator (24), the plasma generator (24) comprising an air ionization discharging electrode (25) and a high-voltage power plug (26), the plasma generator (24) being arranged inside and/or outside the fan-coil air inlet (30), or the plasma generator (24) being arranged between the fan-coil air inlet (30) and the central air-conditioning return air inlet (32), and the high-voltage power plug (26) being connected with the plasma generator (24); and
the sterilization device (7) further comprises the electrostatic negative oxygen ion device (33), the electrostatic negative oxygen ion device (33) comprising at least one group of negative oxygen ion generators (24), an air corona discharging electrode (25) and a high-voltage power plug (26), each of the at least one group of negative oxygen ion generators (24) being arranged inside and/or outside the fan-coil air inlet (30), or the negative oxygen ion generators (24) being arranged between the fan-coil air inlet (30) and the central air-conditioning return air inlet (32), and the high-voltage power plug (26) being connected with the negative oxygen ion generator (24);
the sterilization device (7) further comprises an ultraviolet device (34); wherein the ultraviolet device (34) comprises an ultraviolet lamp tube (27) and a power plug (28) for the ultraviolet lamp tube, the ultraviolet lamp tube (27) being arranged inside and/or outside the fan-coil air inlet (30), or between the fan-coil air inlet (30) and the central air-conditioning return air inlet (32), and the power plug (28) for the ultraviolet lamp tube is connected with the ultraviolet lamp tube (27).
